(19) United States
(12) Patent Application Publication
(10) Pub. No.: US 2009/0087046 A1
(43) Pub. Date: Apr. 2, 2009

(54) Digital blink comparator apparatus and software and methods for operation

(76) Inventor: Matthew Joseph Kuhn, Springfield, IL (US)

Correspondence Address: David Silverstein;Andover IP Law, 44 Park Street Suite 300, Andover, MA, 01810, US

(21) Appl. No.: 11/991,023

(22) PCT Filed: Sep. 5, 2006
(86) PCT No.: PCT/US2006/034479

§ 371 Date: Feb. 26, 2008

Related U.S. Application Data
(60) Provisional application No. 60/715,063, filed on Sep. 8, 2005.

(51) Int. Cl. G06K 9/00; A61B 5/00

(52) U.S. Cl. 382/128; 600/407

(57) ABSTRACT

New techniques, imaging software, and related imaging systems for visualizing and comparing medical images are disclosed. The apparatus of this invention generally comprises a digital imaging system including a visual display device in combination with software adapted so as to enable two or more digital images to be rapidly alternately superimposed on the same display space and displayed to a viewer (FIG. 7). The digital blink comparator apparatus and methods of this invention may be carried out using or be applied to a wide variety of existing medical imaging interpretation devices including, but not limited to, standard PACS, web-based PACS, cardiac imaging, ophthalmologic imaging, and dental imaging.

### FIELD OF THE INVENTION

This invention relates generally to the field of medical imaging technology, more specifically to radiology and related applications, wherein images of a portion of a body, e.g., images produced by X-rays, CAT scans, MRI techniques, and the like, are used for diagnosis, evaluation, or other medical purposes. More particularly, this invention relates to a digital “blink” method of alternately and in rapid succession displaying new and old images, which have been sized and oriented such that the alternately displayed images are superimposed in substantially the same display location, to more readily detect changes in size and number of abnormalities.

The techniques of this invention provide greater accuracy and time efficiencies than are possible to achieve with prior art approaches. Currently, radiologists have no real notion as to when they have completed the time-consuming process of looking for changes or differences in multiple medical images during a radiological evaluation. With the digital blink comparator methods and apparatus of this invention, however, a medical professional can be essentially certain of detecting all of the differences/changes in two or more images, and can detect those differences in a briefer time frame than is possible using existing methods and apparatus in this field.

### BACKGROUND OF THE INVENTION

The discovery of the dwarf planet Pluto was the result of a 22 year effort in which a large area of the night sky was photographed at high magnification with a telescope on two separate occasions. The resulting tens of thousands of images, obtained weeks to months apart from each other, were compared visually. The “fixed” stars were not expected to change their relative positions between the photographs; since planetary objects orbit the sun, their movements were expected to be detected as the planets shifted their positions relative to the surrounding stars.

It was soon appreciated, however, that the human eye could not reliably detect changes from one photographic plate to another. There were simply too many stars on each plate. As a remedy, an analog device that was called a “blink comparator” was developed. This device mechanically alternated one photographic plate with another, presenting the astronomers with a single location to view the two plates, rather than having to move head and eyes from left to right to compare the photographs. Using the blink comparator, any difference in location of an object on the two plates would “blink” in front of the astronomers, drawing immediate attention to the difference between the two images. In this manner, Pluto was discovered by Clyde Tombaugh in 1930.

Despite the advent of digital photography, digital medical imaging devices and advanced computer techniques, the use of a “blink” method of comparing digital medical images has never been proposed or developed. Most modern radiology departments no longer use film for diagnosis; images are now routinely obtained, evaluated, interpreted and stored in a digital fashion using a commercially-available computerized display and storage system known as PACS (Picture Archive and Communication Systems), or a similar computerized image display and storage system.

Also, despite the widespread presence of PACS and ancillary sophisticated computer work stations, there is currently no known practice of comparing two medical images of a region of the body in a dynamic, “blinking” fashion so as to facilitate rapid and accurate visual identification of changes on the images such that the interpreter is not required to turn his head or to move his eyes repeatedly back and forth looking for differences in the images being compared. It therefore remains a challenging but essential element of a radiologist's work to compare a recently obtained set of image data (e.g., a newly obtained set of computerized tomographic images [CT scans]) with a previous set of similar images.

In view of the increasing importance of medical imaging in the diagnosis and treatment of disease, the critical need to compare old and new images to pinpoint any changes, and the very high standards and expectations in the current practice of medicine, which is intolerant of errors, there is an urgent need for a new and improved approach to detecting new lesions, changes in the size of existing lesions, and in similar applications, as well as in improving efficiency and allowing treatment decisions to be based on more accurate diagnostic information.

### COMPARISON WITH THE PRIOR ART

Existing patents and other prior art in this field do not teach or suggest using a “blink” method of comparing new and old digital images by rapidly alternately displaying in a superimposed orientation similar digital image data sets in order to improve lesion detection capabilities, enhance a radiologist's efficiency by finding all of the abnormalities in a shorter time, and indicate when to stop looking because every “blink” has been identified and evaluated.

For example, U.S. Pat. No. 6,427,022 (Craine '022) and U.S. Pat. No. 5,836,872 (Kenet '872), which are incorporated herein by reference, merely compare photographs of the external appearance of new or growing skin lesions with similar prior skin photographs. Skin is an uneven, partially curved surface; however, the photographs of skin, as used in the Craine '022 and Kenet '872 patents, unrealistically portray it as a flat, two-dimensional structure. By contrast, the methods of the present invention compare cross-sectional digital “slices” of three-dimensional masses of living tissue or body regions using various medical imaging techniques, such as MRI, CT, nuclear medicine, X-rays and the like. The digital blink comparator of this invention does not use images of two-dimensional surfaces only. Instead, this invention takes a volume of tissue or a body region and renders images thereof in a cross-sectional format as they are used in actual medical diagnostic mode. Alternatively, this invention can also usefully compare the “collapsed” views produced by standard X-ray imaging. Such volumes of tissue mass or body regions can be substantially identically aligned when comparing the old and new imaging studies. An analogy here would be to compare two unopened decks of cards that lie next to each other. The cards in each deck are in the same exact order. With the power of modern medical imaging techniques, one can visualize each individual card at a certain depth inside a deck, and one can align or orient that card with the comparable (based on card number or its depth in the deck) card in the adjacent deck for an exact comparison. This type of three-dimensional comparison of image “slices” obviously cannot be done with simple surface photographs.

U.S. Pat. No. 5,016,173 (Kenet '173), which is also incorporated herein by reference, likewise only describes an apparatus for monitoring visually accessible surfaces of the body. In addition to comparing skin surfaces, Kenet '173 also describes the possibility of using this apparatus for the evaluation of interior body surfaces such as the cervix, retina and vascular endothelium (which, the patent says, requires additional optical apparati to be visibly accessible). Once again, even if the above technique were practical, it deals only with surface imaging visualization rather than interrogating data from three-dimensional data image sets.

U.S. Pat. No. 4,747,146 (Nishikawa '146), which is also incorporated herein by reference, uses microprocessor technology to determine differences between the image on one sheet of film compared with the image on another sheet of film. This patent does not specifically mention medical images, and it does not use a “blink visualization” technique as in the present invention. By contrast, the present invention facilitates unaided “human eye” detection of changes and differences rather than relying on a computer to detect changes or differences between two images. It is envisioned, however, that an embodiment of the present invention could employ computer-aided detection, and such a system may be utilized to assist the human eye with such “blink” detection.

U.S. Pat. No. 4,404,590 (Mayer '590), which is also incorporated herein by reference, does not employ medical diagnostic images. The invention here lies in the use of a video camera to aid in detecting differences between two images. A video device as in Mayer '590, however, is of little or no benefit in the evaluation of CT or MRI or similar medical images because video imaging results in loss of image detail due to additions to the optical chain (optic coupling loss). Very small changes/differences cannot be detected using this video imaging approach.

U.S. Pat. No. 4,922,909 (Little '909), which is also incorporated herein by reference, also uses a video device to compare images. Little '909 is specifically directed to the use of the invention for ophthalmologic surgery guidance. By contrast, the digital blink comparator of this invention is not primarily meant to be a surgical guidance device. The apparatus and methods of this invention are ordinarily used retrospectively in reviewing medical images, not as a contemporaneous technique to assist with operations, although such surgical guidance applications are also considered to be within the scope of this invention.

To evaluate changes to a human face through the use of cosmetics, U.S. Patent Publication No. US 2002/0181752 (Wallo '752), which is also incorporated herein by reference, teaches a technique that involves photographing the head in a “before” and “after” fashion, and then superimposing the photographs in order to detect changes. Once again, this patent relates only to a comparison of surface features, whereas the present invention does not use simple surface imaging but, instead, evaluates images representing slices of a three-dimensional tissue mass or body region.

U.S. Pat. No. 6,571,003 (Hillebrand '003),which is also incorporated herein by reference, consistent with its title “Skin imaging and analysis systems and methods,” is yet another patent limited to comparative methods of evaluating skin surfaces. This patent does not teach or suggest any method for evaluating the deeper, three-dimensional tissue masses or body regions of a living animal or person. The interior portions of such masses or regions are invisible to a simple photographic camera but are readily imaged in three-dimensions by CT, MRI, ultrasound and similar techniques, for which this invention alone is suited.

Photos of skin surfaces, which are compared by the prior art techniques discussed above, are created by a simple optical step with a camera lens. Skin is not customarily photographed by dermatologists or other physicians even when screening for skin diseases. Such surface photographs are not part of typical medical care. On the other hand, medical images are typically taken as a sequence of slices of a three-dimensional tissue mass or body region using imaging techniques, which produce images that can be computerized, reconstructed, re-processed, re-sized and otherwise manipulated in a highly organized, predictable, reproducible manner.

In accordance with this invention, the technique employs actual medical images of the type that are routinely used for diagnostic purposes. No surface photographs are created. Instead, diagnostic images are compared in a digital fashion.

In the prior art patents discussed above, the photos in at least some cases are taken by non-professional patients of their own skin. By contrast, with this invention, medical images are obtained by a skilled medical professional using sophisticated medical imaging technologies, and those images are subsequently manipulated according to established medical practices.

The digital medical images used in this invention can be resized, angled, oriented and changed in contrast and brightness to better match and align with each other. Such image manipulations are not performed, or even possible or desirable, in the patents referenced above.

In summary, all of the previous patents and literature in this field at most describe taking and comparing photographs of skin surfaces. Prior art techniques do not compare collapsed views or slices, and therefore do not visualize the depths (interiors) of thee-dimensional tissue structures. For comparison with prior art technologies, one may consider the outside surface of a salami. Prior art techniques can only take photographs of the outside of the salami. They cannot look at and evaluate the heterogeneous interior regions of a salami or compare renderings of slices of the salami that can be taken from a full range of angles/vantages. By contrast, medical imaging using the blink comparator techniques of this invention enable one to look at the inside of the salami, to create images of slices of the salami (cut in any direction and at any angle)—even while the salami itself remains uncut in actuality—and to compare similar images taken at different times. Prior art imaging techniques cannot utilize images of the inside of the brain, chest, abdomen, or other body regions, nor can they be utilized to compare older images of tissues inside the body with new images.

### OBJECTS OF THE INVENTION

Accordingly, a general object of the present invention is to provide apparatus, software and methods for comparing two or more digital images.

It is also an object of this invention to provide medical imaging systems utilizing as an element thereof more or less conventional medical imaging technology, in combination with a novel software adaptation of commercially-available software, which allows a series of two or more digital images of a particular three-dimensional body part or region to be alternatively displayed, one image substantially upon the other, and/or aligned with one another, in a rapid fashion, thereby presenting the alternating images to the interpreter/diagnostician in a fashion such that the images rapidly alternate back and forth causing a visually perceptible “blink” effect to identify changes or differences in the images being compared.

Another object of this invention is to provide a digital blink comparator technique and apparatus that easily adapts to most existing medical software imaging applications (such as the PACS systems) by incorporating software adaptations that would be a matter of routine software programming to one of ordinary skill in this art based on the teachings of this application.

Another object of this invention is to provide a digital blink comparator technique and apparatus wherein a generic computer monitor of almost any type, or other types of image display devices, can be adapted to function as the display medium or viewing station for purposes of this invention.

Another object of this invention is to provide a digital blink comparator technique and apparatus wherein virtually any simple desktop computer, or other types of electronic data processing devices, using essentially any of the popular commercially or freely available computer operating systems, such as Microsoft Windows®, Apple Macintosh®, Unix®, Linux®, etc., can be used or easily adapted to rapidly alternately display two or more medical images according to this invention using almost any generic digital photographic applications.

Another object of this invention is to provide a digital blink comparator technique and apparatus wherein relatively minor software modifications, within the skill of a computer-savvy technician, in a computer operating system will allow for alternately displaying two or more images rapidly in sequence in accordance with this invention, such software preferably including or being adapted to provide for a variable and easily adjustable “blinking” rate for carrying out the “blinking” process.

Another object of this invention is to provide a digital blink comparator technique and apparatus wherein, depending upon the viewer's individual style, the modality from which the imaging is derived, and the particular pathology being displayed, the preferable “blink” rate may typically be varied from about 0.2 seconds to about 0.5 seconds; but, in specialized circumstances, wherein slower or faster “blink” rates can be provided, and, in general, wherein the “blink” rate may be varied broadly from about 0.001 seconds (or even faster if technology permits) to about 1 second or even greater.

These and other objects, benefits and advantages of the apparatus and methods of this invention will be better understood from the following description, which is intended to be read in conjunction with the several drawings.

### SUMMARY OF THE INVENTION

In a general embodiment, the present invention is directed to medical imaging systems based on apparatus, software, and related methods of use which incorporate a blink method of rapidly alternating two (or more) digital medical images that are of approximately the same scale and type. Rather than using conventional methods of comparing images in a side-by-side or top-over-bottom fashion, substantially superimposing images in the same display space and rapidly switching between them allows for increased lesion conspicuity and added detection capabilities. The interpreter's senses are heightened, additional areas of the retina are involved, and additional neurons from other parts of the brain are recruited to more accurately identify changes or differences in the images being compared.

With the apparatus, software and methods of this invention, there is no need for constant side-to-side or up/down head movements. Likewise, rapid, darting eye movements are minimized since the images being compared will be presented directly in front of the interpreter.

Existing PACS software (i.e., without being modified according to this invention), which is commercially available from dozens of companies, is currently geared only to view image/data sets in a linear series of images or in stacks of images. Comparison (older) imaging studies may thus be stacked or displayed linearly, such as by “hanging” the images manually or on a computer screen, above or below a current set of images to be visually compared and interpreted, as illustrated for example in FIGS. 3A and 3B and in FIG. 4. At the present time, commercially-available digital imaging software does not provide for viewing or generating comparison studies in a mode of rapidly alternating two or more images at substantially the same display position on a single monitor or other viewing device. Based on the teachings of this invention, however, such a capability can generally be easily incorporated into such software by making relatively minor modifications in the software code of each company's product For example, Microsoft PowerPoint® can be readily adapted to have blink comparator capability by superimposing two comparison images multiple times on a single slide, and by adjusting the transition time electronically or manually to be in a broad range of about one one-thousandth of a second to about one second, preferably in a range of about one-fifth to about one-half of a second, in accordance with this invention.

Experiments have shown that even when two (or more) medical images are of somewhat different scale, or are angled or otherwise somewhat differently oriented in viewing perspective, the digital blink comparator apparatus and methods of this invention can be adapted to work quite well and still produce superior results as compared with conventional side-by-side image comparisons. For example, when the two (or more) images to be compared by the digital blink comparator techniques of this invention are not well aligned in the x or y planes on the display screen, a simple method can be provided for moving an image up, down, left, right, or a combination of the above to at least partially improve image alignment with another image, as illustrated in FIGS. 5A, 5B and 6A, 6B. This can be accomplished, for example, by a simple and familiar computer manipulation knows as the “click and drag” technique. Additionally, in another embodiment of this invention, the images can be digitally aligned if their scales are different, for example by the also familiar computer manipulation technique of clicking on a corner of one image and moving diagonally into the image (if it is too large) or diagonally out of the image (if it is too small). If the two images are of different proportions in height and/or width, a first digital image can be made more similar to a second digital image by clicking on either a side or the top or bottom of the first image and dragging left/right or up/down, to improve alignment, again as illustrated in FIGS. 5A, 5B and 6A, 6B.

Still another image manipulation technique is to optionally digitally rotate one or both images so that they align better if the patient is turned or leaning when one of the images is taken. An additional optional technique to improve the capability of the digital blink comparator apparatus and methods of this invention is to utilize or adapt software so as to superimpose the two images at the same time on the display screen in a semi-transparent manner so that alignments of the two images in all directions can more easily be accomplished. These operations will be recognized by those skilled in the art as familiar computer image manipulation processes, and most, if not all, modem computer operating systems/software applications have, or can be adapted to have, such image manipulation capabilities based on the teachings of this invention.

By rapidly switching the images presented to the human eye at a sufficiently rapid “blink” rate, any changes or differences between the images will be perceived as motion. Such changes in the images displayed might be due to new lesions, growth or diminution in size of a lesion, as well as changes in density, signal intensity and enhancement characteristics, as illustrated in FIG. 7.

These and other aspects and embodiments of this invention will be better understood from the following more detailed description of the invention and the accompanying drawings.

### BRIEF DESCRIPTION OF THE DRAWINGS

FIGS. 1A and 1B schematically show two hypothetical frontal chest radiographic views taken at different times.

FIG. 2 is a schematic representation of superimposing the images of FIGS. 1A and 1B.

FIGS. 3A and 3B are schematic representations of a comparison of two MRI image studies using a manual “hanging” comparison method.

FIG. 4 illustrates a typical “hanging” protocol for MRI image interpretation using a computerized display and storage system.

FIGS. 5A and 5B schematically represent CT or MRI images of a brain section taken at different times.

FIGS. 6A and 6B show the FIG. 5A and FIG. 5B images, respectively, with the FIG. 5B image realigned and re-scaled in accordance with embodiments of this invention in preparation for applying the blink comparator technique of this invention.

FIG. 7 shows a schematic representation of an image produced by applying a digital blink comparator method according to the present invention to the images of FIGS. 6A and 6B.

### DETAILED DESCRIPTION OF PREFERRED EMBODIMENTS

Radiologists face great challenges when going through and trying to compare hundreds (sometimes, thousands) of images per study. This difficulty is compounded by the need to accurately compare the findings on a new study with those of the prior study. It is particularly difficult to identify new changes in images when there are many other background abnormalities. Prior art comparison methods require looking at images that are spaced apart from each other typically by 0.5 meters or more, and repeatedly moving one's eyes up and down or side to side in a tiresome, physically demanding and fatiguing exercise. This can easily result in a physician/diagnostician “missing” lesions or not detecting subtle changes in lesion size or configuration. Because the actual images being compared are not superimposed using conventional practice, but instead are spaced at a distance from each other, the interpreter needs to perform time-intensive measurements on each image to compare sizes and shapes. By contrast with the prior art in this field, this invention applies the principle of a “blink” comparator to the field of medical imaging.

The human eye, similar to that of other organisms, is more sensitive to motion than it is to static images. Physiologically, moving objects incorporate additional areas of the retina and recruit additional neurons in different portions of the brain. From a neuropsychological point of view, visual motion wakes the brain to attention and, in doing so, helps prevent errors that commonly arise from “satisfaction of search” issues (the tendency of a radiologist to stop looking for additional abnormalities once he has found a single lesion or found what was expected, rather than continuing to look for additional lesions or unexpected abnormalities). The digital blink comparator technique of this invention also helps to prevent the tedium that comes from comparing thousands of lesions, as well as adding greater depth and visual understanding into how lesions change over time. In this respect as well, the imaging invention of this application represents a great stride forward in medical technology and diagnostic techniques.

The blink method of alternately and rapidly superimposing new and old images on the same display space readily detects changes in size and number of abnormalities. This provides greater accuracy and time efficiencies. Currently radiologists have to decide arbitrarily as to when they have completed looking for changes, but with the digital blink comparator method of this invention, one can be essentially certain of detecting all of the changes, as well as detecting them in a briefer time frame. The various Figures, as described below, help to illustrate the operation of this invention and how it differs from conventional practice.

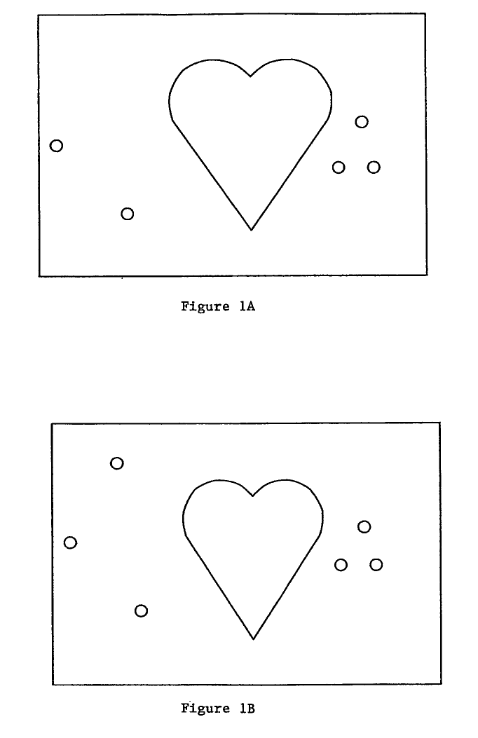

FIGS. 1A and 1B schematically show two hypothetical radiographic views of a frontal chest obtained one year apart. The drawings are placed one above the other to simulate a traditional method of comparative image analysis. The heart is in the center of each drawing and is symbolized by the heart symbol [symbol] . Each chest radiograph contains multiple nodules, each of which is symbolized by a [symbol] . One additional nodule is shown in FIG. 1B which is not present in FIG. 1A.

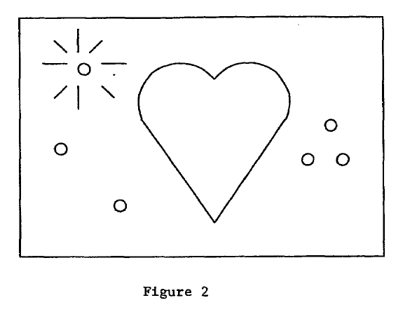

FIG. 2 is a schematic representation of both images (FIGS. 1A and 1B) being alternately displayed in a substantially superimposed orientation on the same display space and being repeatedly and rapidly switched to simulate the digital “blinking” of one image over another in accordance with the present invention. The “new” nodule which appeared in FIG. 1B is now easily identified because this lesion “blinks” on and off as the two images are alternated on the display screen, and the viewer does not have to move his eyes back and forth to try to identify differences in the two images. The “blinking” of the new node is symbolized schematically in FIG. 2 by multiple rays shown emanating from the additional nodule of FIG. 1B.

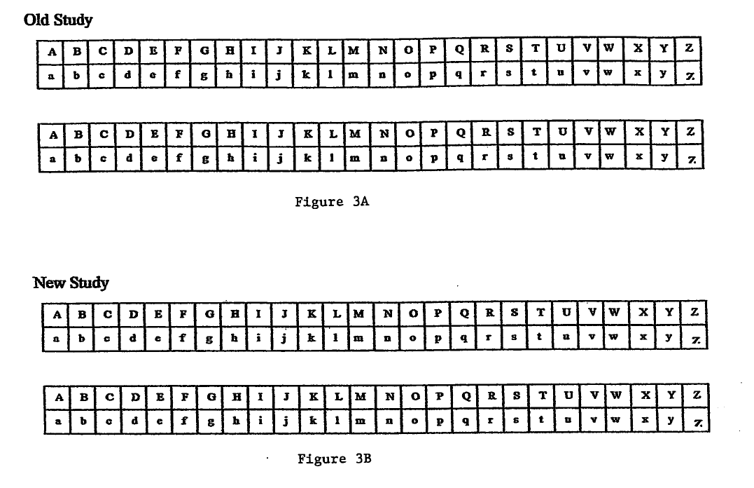

FIGS. 3A and 3B illustrate a conventional, manual approach to displaying and visually comparing two image data sets. FIG. 3A is a schematic representation of a prior MRI study containing 104 “hanging” images (4 rows of 26 images each), which needs to be compared with a new study, FIG. 3B, also with 104 separate images. Each of the 208 letters represents a different, separate individual image (i.e., the same letters in different rows identify different images). Although this type of voluminous data comprising numerous sequential images for comparison is typical for such MR imaging, this manual “hanging” method of display and evaluation presents the interpreter with a tremendous number of images at once and often can overwhelm his visual senses and ability to accurately and completely identify changes or differences between different images.

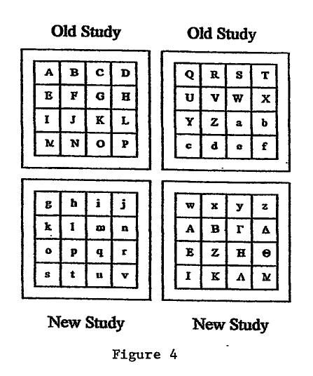

FIG. 4 illustrates a conventional “hanging” image protocol for MRI interpretation using conventional PACS software for image retrieval and display. In this system, which uses four separate monitors or display screens, the old study is divided into two parts and displayed on the top two monitors, while the new study is divided into two parts and displayed on the bottom two monitors. In order for the interpreter to compare the upper and lower corresponding images, he must repeatedly move his eyes and neck to visually compare any two or more images. The images being compared typically are not physically near each other on the display screen (in order to maintain proper sequencing of the images), and it is often difficult under these conditions to diagnose new abnormalities or changes with high reliability.

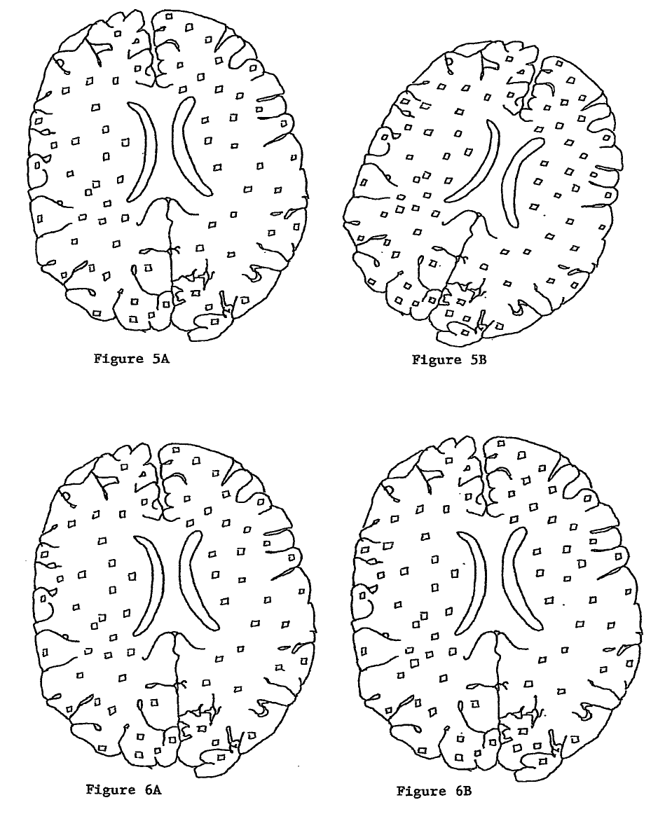

FIG. 5A is a representation of a CT or MRI slice of a brain at a first point in time. Each symbol □ represents a lesion, such as a tumor, stroke or multiple sclerosis plaque. FIG. 5B represents a CT or MRI slice of the same part of the brain obtained on the same patient at a different time, for example one year after the FIG. 5A image was taken. Five new lesions appear on the image of FIG. 5B which were not present one year before on the image of FIG. 5A, but the new lesions are very difficult to identify visually. Among other changes, the patient's head is now turned or tilted to the left in FIG. 5B, and the image obtained for FIG. 5B was smaller than that in FIG. 5A. Using the blink comparator apparatus, software and methods of this invention, however, the image in FIG. 5B can be aligned, oriented, and re-scaled to better match the original image of FIG. 5A. In this manner, two or more digital images can be made to substantially superimpose on one another in the same display space prior to initiating the “blinking” step of rapidly switching the images.

FIG. 6A shows the original image of FIG. 5A. FIG. 6B, however, which is the re-aligned and re-scaled image of FIG. 5B, now more closely matches the image of FIG. 6A in scale and orientation. Comparing FIGS. 6A and 6B visually without the “blinking” step of this invention, however, it can be seen that the five new lesions appearing in FIG. 6B are still very difficult to identify.

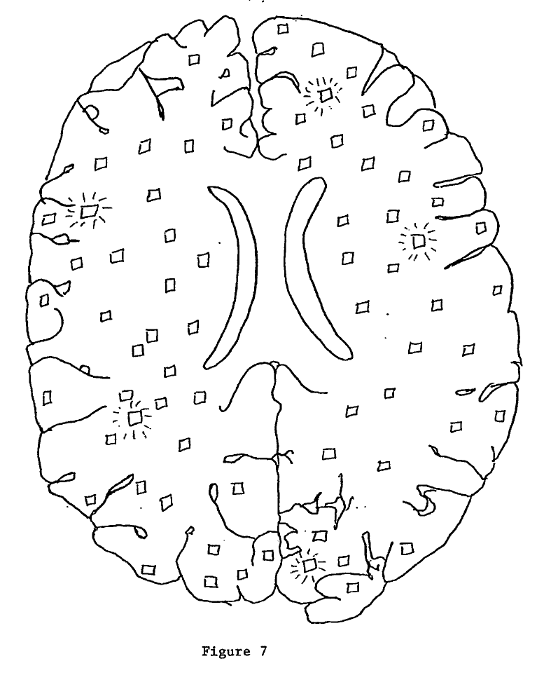

FIG. 7 is a representation of applying the blink comparator apparatus and method according to this invention to FIGS. 6A and 6B. By digitally switching the image of FIG. 6A with that of FIG. 6B on a display screen in front of the interpreter at a frequency of, for example 0.3 seconds, or another suitable “blink” rate, the five new lesions present on FIG. 6B (represented by emanating rays) are now easy to recognize because they “blink” as the images are rapidly switched. The “blink” rate can be varied within a broad range of, for example, about once every 0.001 second to about once every 1 second, by routine experimentation to optimize the “blink” rate for comparing any image data sets. The “blink”. rate (i.e., the rate at which displayed images are switched) must be sufficiently rapid to cause any differences in the images being alternately displayed to create a visually perceptible “blink” effect. This invention thereby makes the interpreter's work more accurate and more time efficient because of the relative ease in visually identifying each “blink.”

Therefore, this concept of applying a digital blink comparison method to medical images differs from, and is far superior to, the prior art imaging technologies discussed above which only used photographs of skin surfaces for comparison.

Radiologists process visual information in a highly individualized, yet highly evolved manner which includes physiological and psychological effects. By adapting the simple analog blink comparator approach that was used historically for analog astronomical photographs to a technique for evaluating digital medical images, the digital blink comparator apparatus and methods of this invention will significantly decrease medical errors. Such reduction in errors would especially include reducing the incidence of failure to notice new lesions or to notice changes in existing lesions.

The above principles, and the related apparatus, software and methods of this invention, can be applied to multiple areas of medical imaging. This includes but is not limited to all areas of diagnostic radiology, nuclear medicine, mammography, cardiac imaging, ophthalmologic imaging and dental imaging.

Examples of specific functionality for the apparatus, software and methods of this invention include: identifying new nodules on a chest radiograph; identifying growth of tumors on CT scans or on MRI images of the brain, neck, chest, abdomen and pelvis; identifying the development of calcified or non-calcified plaques in blood vessels of the body, including the coronary arteries, the carotid arteries, the aorta and other vessels; identifying changes in the retina on static images or fluorescein angiograms over time; and identifying degenerative conditions of teeth. The apparatus and methods of this invention have human medical and dental as well as veterinary applications.

The techniques by which images for use with this invention can be derived include, but are not limited to X-rays, computed tomographic images (CT or CAT scan images), ultrasound and MRI. Simple X-rays produce an image which superimposes all of the soft tissue and bony material that it passes through. The resultant image is a “collapsed view” in which, for a chest X-ray, the breasts, chest wall, lungs and heart are all superimposed upon each other. Cross-sectional imaging cannot be obtained in this manner. CT, ultrasound, MRI, and some nuclear medicine techniques, however, can be used to produce individual “slices” of three-dimensional tissue masses and body regions. Both the collapsed views produced by regular X-rays as well as the individual slices obtained by cross-sectional medical imaging techniques can be usefully compared using the blink comparator techniques and apparatus described herein.

In one preferred embodiment, this invention is directed to a medical diagnostic system comprising in combination an electronic storage medium for storing two or more stored digital images, a display medium for displaying a stored digital image, and digital imaging software adapted to control the display of said digital images on said display medium so as to switch rapidly between displaying at least a first and at least a second of said digital images.

In another preferred embodiment, a medical diagnostic system according to this invention includes stored digital images comprising images representing slices of a three-dimensional body region obtained using computed tomographic images, ultrasound, magnetic resonance imaging, or nuclear medicine techniques, or collapsed images of a three-dimensional body region produced by X-rays.

In another preferred embodiment, a medical diagnostic system according to this invention includes digital imaging software adapted to manipulate a digital image displayed on the display medium in at least one or several of the following ways: (a) alternately displaying in sequence at least two digital images multiple times on the same display screen; (b) adjusting the transition time between the display of different images; (c) moving a first image into alignment with a second image on the display medium, (d) shrinking or enlarging a first image to better align it with a second image; (e) rotating a first image to better align it with a second image; and (f) superimposing at least two images in a semi-transparent manner to assist in image alignment.

In another preferred embodiment, a medical diagnostic system according to this invention provides a digital image blink rate that can be adjusted to be from about 0.001 seconds to about 1 second, or, in a more preferred embodiment, a digital image blink rate adjusted to be from about 0.2 seconds to about 0.5 seconds.

In another preferred embodiment, a medical diagnostic system according to this invention comprises a computer processing unit, a computer monitor, and an operating system in combination with digital imaging software that provides the capability to rapidly and repeatedly switch the display of different digital images. The digital imaging software is preferably a modified Picture Archive and Communications System (PACS) adapted to rapidly and repeatedly switch the display of different digital images.

In another preferred embodiment, this invention is directed to a method of comparing at least two digital images, each representing a similar slice or collapsed view of a three-dimensional body region, which may be taken at different times, said method comprising the steps of: (a) displaying only a first digital image on a display screen for a brief period of time; (b) displaying only a second digital image on the display screen for a brief period of time; and (c) alternately repeating steps (a) and (b) to identify differences between said first and second digital images.

In another preferred embodiment of a method according to this invention, at least the first and second digital images are switched back and forth sufficiently rapidly to cause any differences between them to create visually perceptible blink effects. In a more preferred embodiment, the digital image blink rate ranges from about 0.001 seconds to about 1 second. In an even more preferred embodiment, the digital image blink rate ranges from about 0.2 seconds to about 0.5 seconds.

In another preferred embodiment of a method according to this invention, at least one of the digital images is modified prior to initiating the “blinking” step to make it better align with another digital image in location, size and/or orientation. Such method may comprise one or more of the following digital image manipulation steps:
(i) moving a first image into alignment with a second image on the display screen;
(ii) shrinking or enlarging a first image to better align it with a second image;
(iii) rotating a first image to better align it with a second image; and,
(iv) superimposing at least two images in a semi-transparent manner to assist in image alignment.

In another preferred embodiment of a method according to this invention, the digital images comprise images representing slices of a three-dimensional body region obtained using computed tomographic imaging, ultrasound, magnetic resonance imaging, or nuclear medicine techniques or collapsed images of a three-dimensional body region produced by X-ray imaging.

In another preferred embodiment of this invention, the blink comparator method of this invention may be incorporated into a medical procedure selected from the group consisting of diagnostic radiology, nuclear medicine, mammography, cardiac imaging, ophthalmologic imaging, and dental imaging.

In still another preferred embodiment, this invention is directed to computer software for imaging applications, said software providing for carrying out at least the functions of: (a) retrieving at least a first stored digital image from a storage medium; (b) displaying the first stored digital image on a visual display medium for a short period of time; (c) immediately thereafter retrieving at least a second stored digital image from a storage medium; (d) displaying for a short period of time the second stored digital image on the same visual display medium where the first stored digital image had previously been displayed; and (e) rapidly repeating steps (a), (b), (c) and (d) in sequence. Such computer software provides for step (e) to be repeated sufficiently rapidly to cause any differences between the first stored image and the second stored image to create visually perceptible blink effects.

In a preferred embodiment of computer software according to this invention, the computer software provides for carrying out at least one or more of the following operations:
(a) adjusting the transition time between the display of different images;
(b) moving a first image into alignment with a second image on the display screen;
(c) shrinking or enlarging a first image to better align it with a second image;
(d) rotating a first image to better align it with a second image; and,
(e) superimposing at least two images in a semi-transparent manner to assist in image alignment.

It will be understood and appreciated by those skilled in the art that various changes and modifications may be made in the above-described apparatus, systems, software and methods for digital blink comparator evaluation of digital images without departing from the scope or the spirit of the invention as described herein, and it is intended that all matter contained in the above description and the attached drawings shall be interpreted in an illustrative and not a limiting sense.

Having described the invention, what is claimed is:

1.-22. (canceled)

23. A medical diagnostic system comprising in combination an electronic storage medium storing at least first and second sets of stored digital images, a display medium for displaying a stored digital image, and digital imaging software adapted to control the display of said digital images on said display medium so as to switch rapidly between displaying at least a first image from said first set and at least a second image from said second set of said stored digital images,
wherein said first set of stored digital images comprises images representing slices taken at a first time period at different depths of a three-dimensional body region or tissue structure, said first set of images collectively visualizing at least a portion of the interior of the three-dimensional body region or tissue structure at said first time period, and also wherein said second set of stored digital images comprises images representing slices corresponding respectively to slices in said first set of images both in depth and in the physical orientation of the body region or tissue structure but taken at a second time period.

24. A system according to claim 23 wherein said first and second sets of stored digital images are obtained using computed tomographic images, ultrasound, magnetic resonance imaging, or nuclear medicine techniques, or collapsed images of a three-dimensional body region produced by X-rays.

25. A system according to claim 23 wherein said digital imaging software is adapted to manipulate a digital image displayed on the display medium in at least one of the following ways: (a) alternately displaying in sequence at least two digital images multiple times on the same display screen; (b) adjusting the transition time between the display of different images; (c) moving a first image into alignment with a second image on the display medium; (d) shrinking or enlarging a first image to better align it with a second image; (e) rotating a first image to better align it with a second image; and (f) superimposing at least two images in a semi-transparent manner to assist in image alignment.

26. A system according to claim 25 wherein the digital imaging software is adapted to manipulate a digital image in each of the ways (a) to (f).

27. A system according to claim 23 wherein the digital image blink rate is adjusted to be from about 0.001 seconds to about 1 second.

28. A system according to claim 23 wherein the digital image blink rate is adjusted to be from about 0.2 seconds to about 0.5 seconds.

29. A system according to claim 23 wherein said medical diagnostic system comprises a computer processing unit, a computer monitor, and an operating system in combination with digital imaging software that provides the capability to rapidly and repeatedly switch the display of different digital images.

30. A system according to claim 23 wherein the digital imaging software is a modified Picture Archive and Communications System (PACS) adapted to rapidly and repeatedly switch the display of different digital images.

31. A method of comparing at least first and second sets of digital images to identify differences over time in corresponding images of said first and second sets, wherein said first set of digital images comprises images representing slices taken at a first time period at different depths of a three-dimensional body region or tissue structure, said first set of images collectively visualizing at least a portion of the interior of the three-dimensional body region or tissue structure at said first time period, and also wherein said second set of digital images comprises images representing slices corresponding respectively to slices in said first set of images both in depth and in the physical orientation of the body region or tissue structure but taken at a second time period, said method comprising the steps of:
(a) displaying only a first digital image from said first set of images on a display screen for a brief period of time; (b) displaying only a second digital image from said second set of images on the display screen for a brief period of time; and (c) alternately repeating steps (a) and (b) to identify differences between said first and second digital images.

32. A method according to claim 31 wherein the first and second digital images are switched back and forth sufficiently rapidly to cause any differences between them to create visually perceptible blink effects.

33. A method according to claim 31 wherein the digital image blink rate ranges from about 0.001 seconds to about 1 second.

34. A method according to claim 31 wherein the digital image blink rate ranges from about 0.2 seconds to about 0.5 seconds.

35. A method according to claim 31 wherein at least one of the digital images is modified prior to step (c) to make it better align with another digital image in location, size and/or orientation.

36. A method according to claim 31 further comprising at least one of the following digital image manipulation steps:
(i) moving a first image into alignment with a second image on the display screen;
(ii) shrinking or enlarging a first image to better align it with a second image;
(iii) rotating a first image to better align it with a second image; and,
(iv) superimposing at least two images in a semi-transparent manner to assist in image alignment.

37. A method according to claim 36 comprising at least two of the image manipulation steps.

38. A method according to claim 31 wherein said first and second digital images each represent a similar slice or collapsed view of a three-dimensional body region.

39. A method according to claim 38 wherein said first and second digital images were taken at different times.

40. A method according to claim 31 wherein said digital images comprise images representing slices of a three-dimensional body region obtained using computed tomographic imaging, ultrasound, magnetic resonance imaging, or nuclear medicine techniques or collapsed images of a three-dimensional body region produced by X-ray imaging.

41. A method for carrying out a medical procedure selected from the group consisting of diagnostic radiology, nuclear medicine, mammography, cardiac imaging, ophthalmologic imaging, and dental imaging, said method including the steps of comparing at least two digital images according to the method of claim 31.

42. Computer software for imaging applications, said software providing for carrying out at least the functions of: (a) retrieving at least a first stored digital image from a first set of stored digital images stored on a storage medium, wherein said first set of stored digital images comprises images representing slices taken at a first time period at different depths of a three-dimensional body region or tissue structure, said first set of images collectively visualizing at least a portion of the interior of the three-dimensional body region or tissue structure at said first time period; (b) displaying the first stored digital image on a visual display medium for a short period of time; (c) immediately thereafter retrieving at least a second stored digital image from a second set of stored digital images stored on a storage medium, wherein said second set of stored digital images comprises images representing slices corresponding respectively to slices in said first set of images both in depth and in the physical orientation of the body region or tissue structure but taken at a second time period; (d) displaying for a short period of time the second stored digital image on the same visual display medium where the first stored digital image had previously been displayed; and (e) rapidly repeating steps (a), (b), (c) and (d) in sequence.

43. Computer software according to claim 42 providing for step (e) to be repeated sufficiently rapidly to cause any differences between the first stored image and the second stored image to create visually perceptible blink effects.

44. Computer software according to claim 42 providing for carrying out at least one of the following operations:
(a) adjusting the transition time between the display of different images;
(b) moving a first image into alignment with a second image on the display screen;
(c) shrinking or enlarging a first image to better align it with a second image;
(d) rotating a first image to better align it with a second image; and,
(e) superimposing at least two images in a semi-transparent manner to assist in image alignment.
